(19) United States
(12) Patent Application Publication
(10) Pub. No.: US 2022/0082246 A1
(43) Pub. Date: Mar. 17, 2022

(54) LIGHTING APPARATUS FOR A CONTAINER DEVICE OF A HOUSEHOLD APPLIANCE

(71) Applicant: emz-Hanauer GmbH & Co. KGaA, Nabburg (DE)

(72) Inventors: Manfredi SIGNORINO, Wackersdorf (DE); Martin BRABEC, Nabburg (DE); Matthias WALLNER, Pfreimd (DE)

(73) Assignee: emz-Hanauer GmbH & Co. KGaA, Nabburg (DE)

(21) Appl. No.: 17/411,776

(22) Filed: Aug. 25, 2021

(30) Foreign Application Priority Data
Sep. 17, 2020 (DE) 102020124237.9

(51) Int. Cl. F21V 33/00; A61L 2/08

(52) U.S. Cl. CPC F21V 33/0044; F21Y 2115/10; A61L 2/084

(57) ABSTRACT

The invention relates to a lighting apparatus for a container device of a household appliance, in particular a washing machine, wherein at least one disinfection apparatus is integrated in the lighting apparatus, wherein the at least one disinfection apparatus comprises a first radiation source device.

### CROSS-REFERENCE TO RELATED APPLICATION

This application claims the benefit of German Patent Application No. 10 2020 124 237.9 filed Sep. 17, 2020, the entire contents of which are hereby incorporated herein by reference.

### FIELD

The invention relates to a lighting apparatus for a container device of a household appliance and to a household appliance comprising a container device and a lighting apparatus.

### BACKGROUND

Such a household appliance is, for example, a washing machine or a dishwasher. The container device is what is known as a suds container in which the objects to be washed and the washing liquid are located. When it comes to washing machines, a distinction is made between top-loaders, where the loading hatch is on the top, and front-loaders, where a porthole on the front usually serves as a loading hatch. Lighting is often provided in the container device in order to facilitate loading and unloading.

As the operating time increases, it is often desirable for the container device to be disinfected in order to remove microorganisms such as bacteria, yeasts and moulds. Furthermore, disinfection of the items to be washed could also be desirable.

### SUMMARY

The object of the present invention is to provide a lighting apparatus for a household appliance which overcomes the disadvantages mentioned. The object of the invention is also to provide a corresponding household appliance.

The object is achieved by the subject matter of claims 1 and 7. The dependent claims comprise preferred embodiments.

According to the invention, a lighting apparatus for a container device of a household appliance, in particular a washing machine, is provided, wherein at least one disinfection apparatus is integrated in the lighting apparatus, wherein the at least one disinfection apparatus comprises a first radiation source device.

By integrating the disinfection apparatus in the lighting apparatus, a single apparatus is provided which can achieve two tasks in an effective manner. This provides a compact apparatus which can be easily accommodated in the household appliance.

According to a preferred embodiment, the first radiation source device comprises at least one radiation source. The at least one radiation source is preferably a light-emitting diode (LED). The first radiation source device preferably emits electromagnetic radiation having a wavelength in a range from 280 nm to 100 nm, more preferably in a wavelength range from 270 nm to 275 nm. Radiation of this type is referred to as UV-C radiation. This radiation has a strong bactericidal effect. The very high-energy UV-C radiation triggers a photochemical reaction. This is absorbed by the cell nucleic acid and, depending on the radiation dose, leads to the destruction or damage of the bacteria and fungal spores. It is absorbed by the DNA of the microorganisms and destroys their structure there. The living cells are inactivated in this way. The UV-C radiation can therefore be used for the targeted damage or inactivation of microorganisms (such as bacteria/viruses, yeasts and moulds). The mechanism of action of UV disinfection has a purely physical basis. UV disinfection does not leave any undesirable chemical compounds or toxic compounds behind. Furthermore, no resistance is caused. UV disinfection is also effective for germs that have already developed resistance to conventional disinfection measures such as alcohol. By means of the first radiation source device, the inner surface of the container device can thus advantageously be disinfected by a corresponding irradiation thereof. The air in the container device can also be cleaned or disinfected. Unpleasant odours in the container device can thus be effectively removed.

According to a further preferred embodiment, the lighting apparatus comprises a second radiation source device. The second radiation source device preferably emits electromagnetic radiation which is used to illuminate the container device. Preferably, the second radiation source device comprises at least one radiation source. The at least one radiation source is preferably a light-emitting diode (LED). It is advantageous here that the second radiation source device emits electromagnetic radiation in a wavelength range between 400 nm and 700 nm. Accordingly, the second radiation source device preferably emits visible light. The second radiation source device can serve to illuminate the container device, whereby the loading and/or unloading process is facilitated. Such lighting can also have an aesthetic effect. For example, front-loading washing machines comprise a closure apparatus in the form of a porthole, which typically consists of a transparent material, for example glass or a plastics material. The illumination of the container device can therefore advantageously be seen through such a porthole.

According to a further preferred embodiment, the first radiation source device and the second radiation source device are arranged on a carrier device. The carrier device is preferably designed as a printed circuit board (PCB). Such an advantageous embodiment provides a compact lighting apparatus by means of which the container device can be at least partially irradiated or illuminated both by the first radiation source device and by the second radiation source device. The carrier device is preferably plate-like. The carrier device preferably has a substantially circular design.

According to a further preferred embodiment, the first radiation source device and the second radiation source device are arranged on the front of the carrier device. At least one cooling device is preferably arranged on the back of the carrier device. A heat sink is preferably arranged on the back of the carrier device. The first radiation source device and the second radiation source device are preferably not operated simultaneously or only for a short time. In this way, sufficient thermal efficiency of the lighting apparatus can be ensured and the cooling device can have smaller dimensions. The first radiation source device advantageously comprises only one radiation source. The only one radiation source is preferably arranged substantially centrally on the carrier device. The term “substantially” should be construed as including minor tolerance deviations. This should also apply accordingly to other uses of the term “substantially.” The second radiation source device advantageously comprises at least two radiation sources. The second radiation source device preferably comprises at least three radiation sources. It is advantageous here that the at least two radiation sources are arranged on the carrier device radially at a distance from the radiation source of the first radiation source device. With such an arrangement of the first radiation source device and the second radiation source device, the heat produced by the radiation sources can be effectively transported away from the cooling device or the heat sink. This ensures stable operation of the first radiation source device and the second radiation source device. The radiation source of the first radiation source device is preferably not exposed to a temperature higher than 80° C. as a result of the cooling device or the heat sink.

According to a further advantageous embodiment, the first radiation source device and the second radiation source device are arranged in a housing. The housing preferably comprises a window which is transparent both to the electromagnetic radiation from the first radiation source device and to the electromagnetic radiation from the second radiation source device. The window is preferably made of quartz glass, which is transparent to UV-C radiation. Quartz glass is a glass which, in contrast to conventional glasses, does not contain any additions of sodium carbonate or calcium oxide, i.e. consists of pure silicon dioxide (SiO₂). Quartz glass is transparent to electromagnetic radiation, from the ultraviolet wavelength range to the infrared wavelength range. Ordinary window glass and many plastics materials that are transparent to visible light are not transparent to UV-C radiation.

A sealing device is preferably provided between the housing and the window so that the housing seals off the components arranged therein and protects them from splashing water on the container device.

A first driver device is preferably provided for the at least one radiation source of the first radiation source device in the form of a UV LED. A second driver device is preferably provided for the at least one radiation source of the second radiation source device in the form of an LED. A driver device supplies a constant current to the LED and is usually connected in series to the LED. The brightness of the LED can advantageously be adjusted by means of a driver device. Pulse width modulation or linear actuation can preferably be provided for this purpose. The first driver device and/or the second driver device are preferably arranged outside the housing of the lighting apparatus in the household appliance. It would also be conceivable that the first driver device and/or the second driver device are arranged within the housing of the lighting apparatus.

The present object of the invention is also achieved by a household appliance comprising at least one lighting apparatus. The household appliance can be equipped with all the features already described above for the lighting apparatus, individually or in combination with each other, and vice versa.

Such a household appliance advantageously comprises a housing and a container device provided therein. Such a household appliance can advantageously be a washing machine or a dishwasher. The container device can also be referred to as a suds container. The items to be washed are placed in this suds container. During the washing process, a washing liquid in the form of water mixed with a treatment agent is then introduced into the suds container.

The household appliance can comprise only one lighting apparatus or a plurality of, for example, two lighting apparatuses.

According to a preferred embodiment, the housing of the household appliance comprises a closure apparatus by means of which it can be closed. The housing of the household appliance is preferably designed in a cubic or cuboid shape. The household appliance preferably has at least three side walls. The housing can comprise two lateral side walls. The housing can also comprise a rear side wall. Finally, the housing can comprise a front side wall. It is also advantageous for the household appliance to have a closure apparatus by means of which the container device can be closed. The closure apparatus can be designed, for example, as a door which is integrated in the front side wall or is provided instead of a front side wall. Operating elements for the user can optionally be provided on the front side wall and/or on the closure apparatus. Operating elements of this type are, for example, program selection switches.

According to a preferred embodiment, at least one control device is provided. The control device can be assigned to the lighting apparatus or to the household appliance. In a first state of the lighting apparatus, the first radiation source device or the disinfection apparatus is preferably activated. In a second state of the lighting apparatus, the first radiation source device or the disinfection apparatus is preferably deactivated. In a third state of the lighting apparatus, the second radiation source device is preferably activated. In a fourth state of the lighting apparatus, the second radiation source devices are preferably deactivated. The defined first state and second state are independent of the defined third and fourth state. The lighting apparatus can accordingly be in the first and third state or in the first and fourth state. Likewise, the lighting apparatus can accordingly be in the second and third state or in the second and fourth state. In other words, the first radiation source device or the disinfection apparatus can be activated or deactivated independently of the second radiation source device. Likewise, the second radiation source device can be activated or deactivated independently of the first radiation source device or the disinfection apparatus. The activation and/or deactivation of the first radiation source device or the disinfection apparatus is advantageously carried out by actuation of the first driver device by the control device. The activation and/or deactivation of the second radiation source device is advantageously carried out by actuation of the second driver device by the control device.

According to a further advantageous embodiment, a first status signal relating to the first state of the lighting apparatus can be received or generated by the control device. Preferably, a second status signal relating to the second state of the lighting apparatus can be received or generated by the control device. Preferably, a third status signal relating to the third state of the lighting apparatus can be received or generated by the control device. Preferably, a fourth status signal relating to the fourth state of the lighting apparatus can be received or generated by the control device.

According to a further advantageous embodiment, an input device is provided by means of which the status signals can be generated. The input device preferably sends the status signals to the control device. After receiving the status signal in each case, the control device preferably initiates the corresponding state of the lighting apparatus. The input device can preferably be operated manually. Accordingly, the input device can comprise buttons and/or switches and/or a touch screen, for example. It would also be conceivable for the input device to be suitable for processing voice inputs. Alternatively or cumulatively, the input device could also comprise gesture recognition for inputting commands by means of gestures. The input device can preferably receive the status signals from an external communications device of a user. The connection between the external communications device and the input device is preferably a wireless connection. An external communications device can be a smartphone, a tablet computer, a laptop or a similar device, for example. A corresponding wireless connection can be, for example, an RFID (radio-frequency identification) connection, an NFC (near-field communication) connection, a Wi-Fi connection or a mobile phone connection. Of course, further wireless connections or wired connections can also be used. Accordingly, the user can activate or deactivate the lighting apparatus or the first and/or second radiation source device in a very convenient way, for example via the smartphone.

The activation or deactivation of the lighting apparatus can also advantageously take place automatically, however. For this purpose, the control device itself preferably generates the status signal in each case. The status signals are then processed by the control device in such a way that the corresponding state is initiated. The at least one control device preferably generates the status signals on the basis of sensor data from a sensor device. The sensor device preferably comprises at least one sensor which detects an opening and/or a closure of the closure apparatus. Since UV-C radiation can be dangerous for humans, it is advantageous if the control device only activates the first radiation source device or disinfects the container device when a sensor signal is received from the sensor device, which indicates that the closure apparatus of the household appliance is closed. The second radiation source device is preferably activated when the control device receives a sensor signal which indicates that the closure apparatus of the household appliance is open. Accordingly, the user is provided with lighting for the container device, which facilitates loading and unloading of the container device. It would also be conceivable for the second and/or first radiation source device to be activated when a washing program is carried out. The two radiation source devices could preferably be activated alternately in order to ensure thermal efficiency of the lighting apparatus. Disinfection could preferably be carried out between the individual steps of a washing cycle. The lighting could preferably be activated during the washing cycles in order to provide the household appliance with a correspondingly aesthetic appearance.

The sensor device preferably comprises at least one sensor which detects the loading state in the container device. Such a sensor can, for example, be a weight sensor which detects the weight of the articles introduced. A sensor in the form of a camera system, which can recognise a loading state, for example by image recognition, would also be conceivable. Advantageously, the first state could be triggered or the first radiation source device could be activated when a load is detected, for example in the form of laundry to be cleaned in the case of a washing machine. Alternatively or cumulatively, the sensor device preferably comprises at least one sensor which detects certain gases in the air in the container device. Such gases may be those which cause an unpleasant odour, for example. Preferably, the first state could be triggered or the first radiation source device could be activated when such a gas is detected. After the removal of the gas, the second state could then be triggered or the first radiation source device could be deactivated.

According to a further advantageous embodiment, the household appliance is a washing machine. The household appliance is preferably a front-loading washing machine. A washing machine of this type comprises a container device in the form of a rotatable suds container, which is also called a drum. The container device is closed by a closure apparatus in the form of a porthole. The porthole is advantageously made of a material that is transparent to visible light, such as glass or plastics material. However, this material is advantageously not transparent to UV-C radiation. Thus, when the closure apparatus is closed, no radiation from the first radiation source device can escape from the container device.

According to a further advantageous embodiment, the household appliance comprises a sealing apparatus which is arranged between the container device and the housing. The lighting apparatus is preferably arranged in or on the sealing apparatus and/or integrated therein. By such an arrangement, the lighting apparatus is arranged within the housing of the household appliance in such a way that an at least partial irradiation of the interior of the container device or an at least partial irradiation of an inner wall of the container device can take place. The inner wall of the container device can accordingly be closed throughout.

According to a further advantageous embodiment, in the first state of the lighting apparatus, the first radiation source device irradiates a portion of the container device. The portion of the container device is preferably defined by an angle of incidence of the first radiation source device. The household appliance advantageously comprises a drive apparatus by means of which a rotation of the container device can be driven. The container device is preferably rotated in the first state. As a result of the rotation of the container device, the entire inner wall of the container device or the entire inner surface of the inner wall passes through the irradiated portion. Accordingly, after a rotation through a certain angle of rotation, the entire inner surface of the inner wall has been advantageously exposed to the irradiation. In the first state, there is advantageously a predetermined number of rotations of the container device in order to allow sufficient irradiation of the inner surface. The speed of rotation and the number of rotations are advantageously dependent on the radiation dose or the output power of the first radiation source device. The control device advantageously controls the drive device accordingly in the first state.

According to a further advantageous embodiment, the first state of the lighting apparatus can only be initiated after at least one sensor of the sensor device has detected the closure of the closure apparatus. The control device receives the sensor data and accordingly generates the first status signal relating to the first state of the lighting apparatus. It is also advantageous that the drive apparatus is only activated or the rotation of the container device only takes place in combination with the activation of the first radiation source device after the closure of the closure apparatus has been detected by at least one sensor of the sensor device. Accordingly, the control device controls the drive apparatus after receiving the sensor data. Accordingly, it is advantageous if the container device and/or the objects located in the container device are not disinfected until the closure apparatus is closed. The closure apparatus is, for example, a door, preferably made of a material that is not transparent to the UV-C radiation. The user and/or the surroundings of the household appliance are therefore not exposed to UV-C radiation. Corresponding user safety can thus be ensured.

According to a further preferred embodiment, the first state is activated when the container device is empty. Accordingly, the container device, in particular the inner surfaces of the container device and/or the air in the container device, is advantageously disinfected. The first state of the lighting apparatus or the first radiation source device or the disinfection apparatus is advantageously activated automatically at predetermined times and/or at regular intervals.

According to a further preferred embodiment, the first state is activated in a loaded state of the container device. Accordingly, objects to be washed in the container device, for example items of laundry, are advantageously disinfected. Advantageously, the container device, in particular the inner surfaces of the container device and/or the air in the container device, is also disinfected. The first state is preferably activated before a washing program. Accordingly, the objects to be washed can already be disinfected before the washing cycle is carried out. Alternatively or cumulatively, the first state can also be activated after or during the execution of a washing cycle. This can be particularly important when, in a washing machine, the washed and still damp laundry remains in the container device for some time after the washing cycle. Microorganisms such as bacteria, moulds, etc. can develop in damp textiles in particular.

A status signal can preferably also be generated or received when a specific program of the household appliance is started, for example when a washing program is started. This can be done using the control device or also a further control device.

According to a further advantageous embodiment, a timer device is provided. Such a timer device can preferably be integrated in the control device or also provided as a further device in the household appliance. The status signals can advantageously be generated on the basis of a predetermined point in time or a predetermined time interval. The point in time of the activation of the states can thus advantageously be specified.

The control device advantageously comprises a memory device in which specific sequence programs are stored. Such sequence programs can comprise the sequential actuation of certain devices, such as the first radiation source device, the second radiation source device, etc. Likewise, the intensity of the actuation of these devices can advantageously be provided in such a sequence program. The emitted radiant power, for example, can be controlled by the advantageous control of the operating current of the radiation source devices.

The present object of the invention is also achieved by a method for controlling a household appliance or a lighting apparatus. The method can be equipped with all the features already described above for the apparatus, individually or in combination with each other, and vice versa.

### BRIEF DESCRIPTION OF THE DRAWINGS

Further advantages, aims and properties of the present invention will be explained with reference to the following description of the accompanying drawings. Similar components may have the same reference signs in the various embodiments.

In the drawings:

FIG. 1 is a top view of a lighting apparatus according to one embodiment;

FIG. 2 is a sectional view of a lighting apparatus according to one embodiment;

FIG. 3 shows a container device having a lighting apparatus according to one embodiment;

FIG. 4a, 4b are thermal images of the lighting apparatus according to one embodiment;

FIG. 5 shows a household appliance according to one embodiment;

FIG. 6 shows a household appliance according to one embodiment;

FIG. 7 is a schematic circuit diagram of a household appliance according to one embodiment.

### DETAILED DESCRIPTION

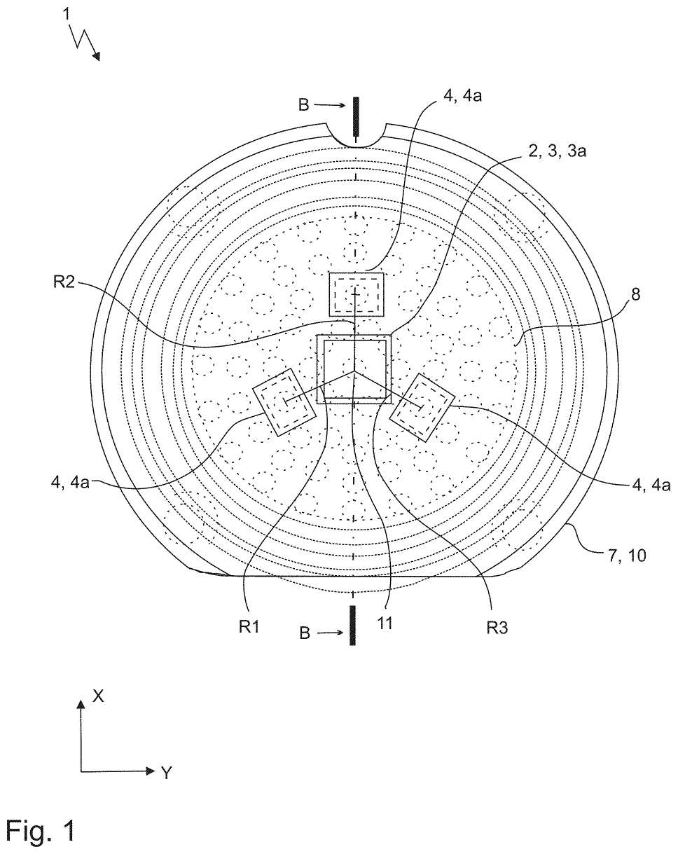

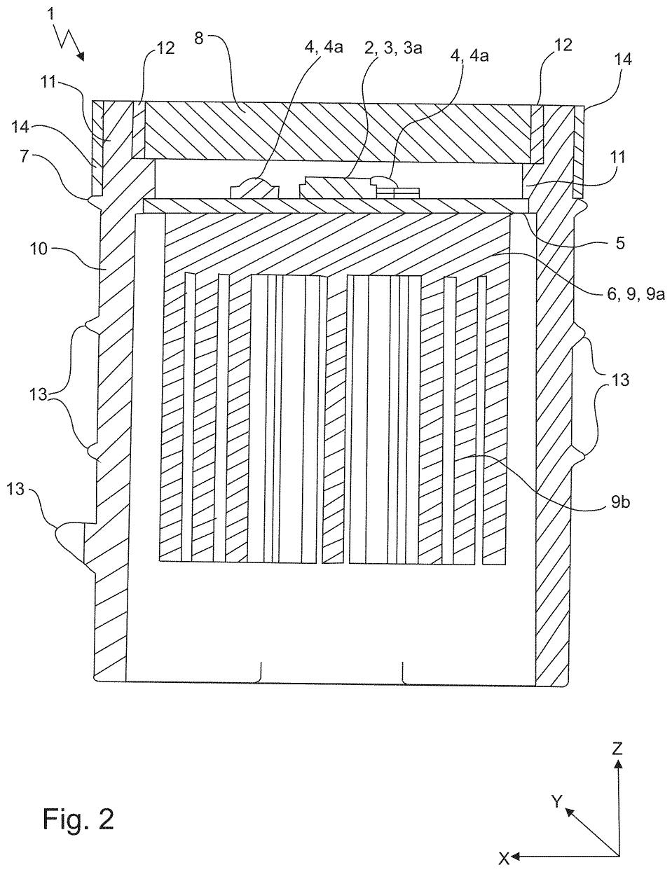

In FIGS. 1 to 4b, a lighting apparatus 1 for a container device 102 of a household appliance 100, in particular a washing machine, is shown, wherein at least one disinfection apparatus 2 is integrated in the lighting apparatus 1, wherein the at least one disinfection apparatus 2 comprises a first radiation source device 3.

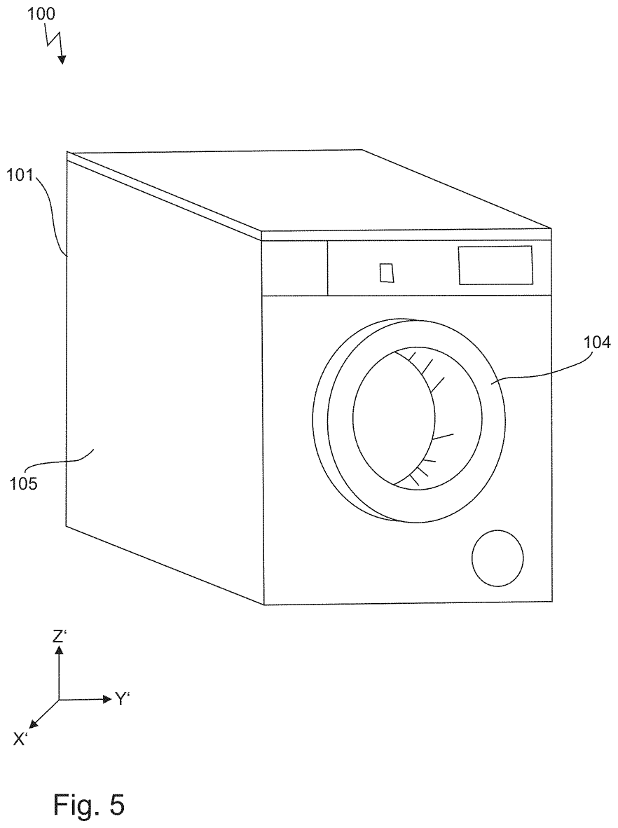

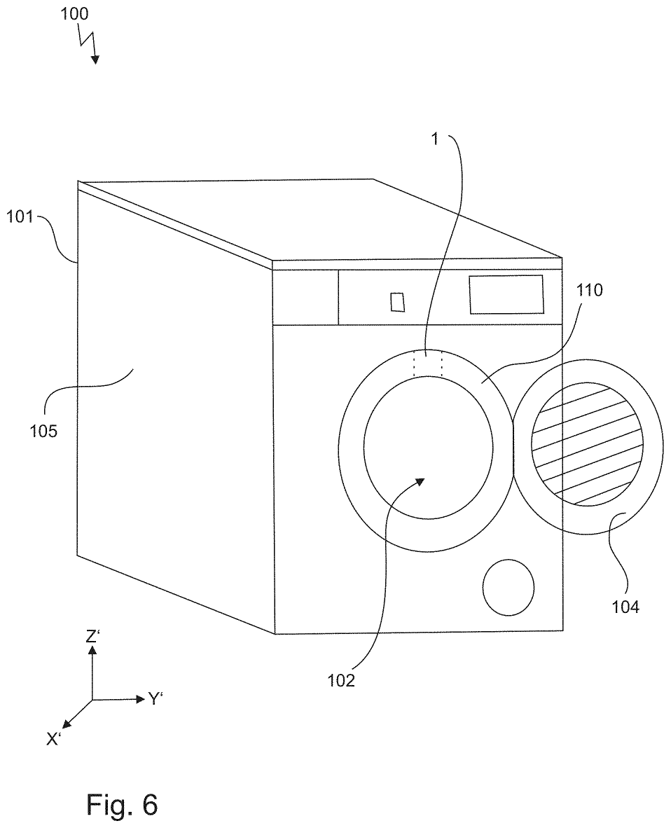

In FIGS. 5 and 6, a household appliance 100 comprising at least one lighting apparatus 1 is shown. The household appliance 100 extends along a height axis Z′, a longitudinal axis X′, and a width axis Y′ and comprises a housing 101 and a container device 102 provided therein.

The first radiation source device 3 comprises at least one radiation source 3a in the form of a light-emitting diode (LED). The first radiation source device 3 emits electromagnetic radiation having a wavelength in a range between 280 nm and 100 nm, what is known as UV-C radiation. The radiation source 3a is thus a UV-C LED.

The lighting apparatus 1 comprises a second radiation source device 4. The second radiation source device 4 emits electromagnetic radiation which is used for irradiating and/or illuminating the container device 102. The second radiation source device 4 comprises at least one radiation source 4a in the form of a light-emitting diode (LED). The second radiation source device 4 emits electromagnetic radiation in a wavelength range between 400 nm and 700 nm, i.e. visible light.

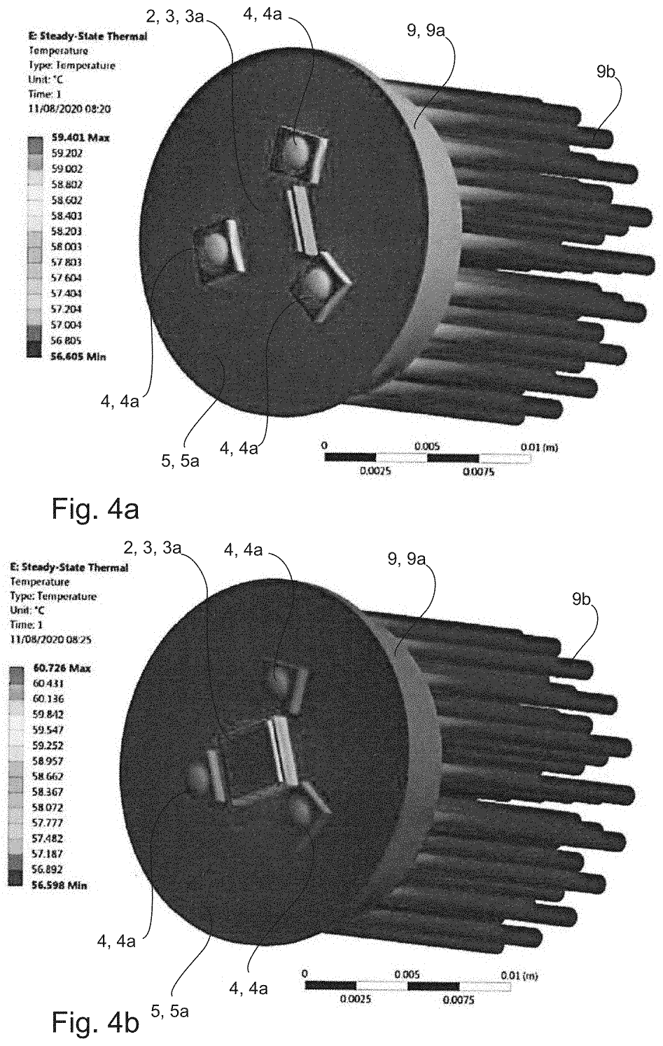

The first radiation source device 3 and the second radiation source device 4 are arranged on a carrier device 5. The carrier device 5 is plate-like and substantially circular. The carrier device 5 is preferably a printed circuit board. This is clearly visible in FIGS. 1, 4a, and 4b. The carrier device 5 has a front 5a and a back 5b. The first radiation source device 3 and the second radiation source device 4 are arranged on the front 5a of the carrier device 5. At least one cooling device 6, preferably a heat sink 9, is arranged or attached on the back 5b of the carrier device 5. The heat sink 9 has a contact portion 9a which makes contact with the carrier device 5. This contact is designed in such a way that the greatest possible heat flow can take place through it. A heat-conducting paste or the like is advantageously provided between the carrier device 5 and the contact portion 9a. The contact portion 9a is advantageously also circular and has substantially the same diameter as the carrier device 5. A multiplicity of cooling fins 9b extend from the contact portion 9a. These cooling fins are rod-shaped and extend, starting from the contact portion 9a, along the height axis Z. In FIGS. 4a and 4b, thermal images are shown in which the heat distribution in the heat sink is shown. In FIG. 4a, only the second radiation source device 4 or the LED emitting visible light 4a are operated. In FIG. 4b, only the first radiation source device 3 or the UV-C LED 3a is operated. The first radiation source device 3 the second radiation source device 4 are preferably not operated simultaneously or only for a short time. In this way, a sufficient thermal efficiency of the lighting apparatus can be ensured and the heat sink 9 can have smaller dimensions, whereby a more compact design of the lighting apparatus can be achieved.

The first radiation source device 3 comprises only one radiation source 3a, which is arranged substantially centrally on the front 5a of the carrier device 5. The second radiation source device 4 comprises at least three, preferably exactly three, radiation sources 4a. The three radiation sources 4a of the second radiation source device 4 are arranged radially at a distance from the radiation source 3a of the first radiation source device 3 on the carrier device 5. The radiation source 3a of the first radiation source device 3 can advantageously be arranged substantially in the centre M of the carrier device. The first radiation source 4a of the second radiation source device 4 can be arranged at a first radial distance R1 from the centre point. The second radiation source 4a of the second radiation source device 4 can be arranged at a second radial distance R2 from the centre point. The third radiation source 4a of the second radiation source device 4 can be arranged at a third radial distance R3 from the centre point. The radial distances R1, R2 and R3 can be the same. It would also be conceivable that the radial distances R1, R2 and R3 are different, or that two of the three radial distances are the same. In FIGS. 1, 4a and 4b it can be seen that the three radiation sources 4a of the second radiation source device 4 are arranged in a triangular manner.

The first radiation source device 3 and the second radiation source device 4 are arranged in a housing 7. The carrier device 5 and the cooling device are also arranged in this housing 7. The housing 7 comprises a window 8 which is transparent both to the electromagnetic radiation of the first radiation source device 3, namely the UV-C radiation, and to the electromagnetic radiation of the second radiation source device 4, namely the visible light. The window 8 preferably consists of a quartz glass. The housing is designed to be substantially cylindrical, preferably circular-cylindrical. Accordingly, the window 8 is also substantially circular, as can be seen in FIG. 1. The housing 7 comprises a main body 10, which is designed as a hollow cylinder having a preferably circular base area. The main body 10 is advantageously made of a plastics material or a metal. At its front end along the vertical axis Z, the main body 10 comprises an opening 10a in which the window is arranged. At its rear end lying along the vertical axis Z, the main body 10 is preferably closed. Preferably, openings for control lines etc. can be provided in the housing 7.

The main body 10 of the housing 7 comprises a wall 10b. On an inner surface of the wall 10b, an inwardly protruding projection 11 is provided, which is preferably circumferential along the entire inner circumference or is arranged in portions along the inner circumference. The projection 11 is arranged along the height axis Z between the window 8 and the carrier device 5. The window 8 preferably rests on a front portion of the projection 11 lying along the height axis Z. The carrier device 5 rests against a rear portion of the projection 11 lying along the height axis Z. The carrier device 5 is preferably fastened to the projection and/or the wall 10b by means of a form-fitting, force-fitting and/or material-fitting connection. This can, for example, be a screw connection, a snap connection or the like. Accordingly, the front 5a of the carrier device 5 equipped with the radiation devices 3, 4 is arranged opposite the window 8 and at a distance from it. A sealing device 12 is provided between the wall 10b of the main body 10 and the window 8. Furthermore, a compression element 14 is provided on the outer surface of the main body 10, by means of which a front portion of the wall 10b can be pressed against the sealing device 12 or the window. The sealing device 12 is thus compressed in the radial direction between the wall 10b and the window 8. As a result, the housing 7 is sealed off. The wall 10b of the main body 10 furthermore comprises mounting projections 13 on an outer surface, which are used to mount the housing 7 in the household appliance and to ensure that the housing is held securely. Furthermore, further sealing elements can be provided on the housing, which are intended to ensure a sealing closure between the housing 7 and the household appliance 100.

In FIGS. 5 and 6, a household appliance 100 is shown in the form of a front-loading washing machine. The housing 101 of the household appliance 100 can comprise a closure apparatus 104, for example a door, by means of which the container device 102 can be closed. Such a door is also known as a porthole. The household appliance 100 can have a substantially cubic or cuboid shape and comprise two lateral side walls 105 and a rear side wall 105 or also a back wall, which is preferably opposite the closure apparatus 104. Finally, the housing can comprise a front side wall 105. The closure apparatus 104 can be designed, for example, as a door which is integrated in the front side wall 105 or is provided instead of a front side wall 105. Operating elements for the user can optionally be provided on the front side wall and/or on the closure apparatus 104. Such operating elements are program selection switches in the case of washing machines, for example.

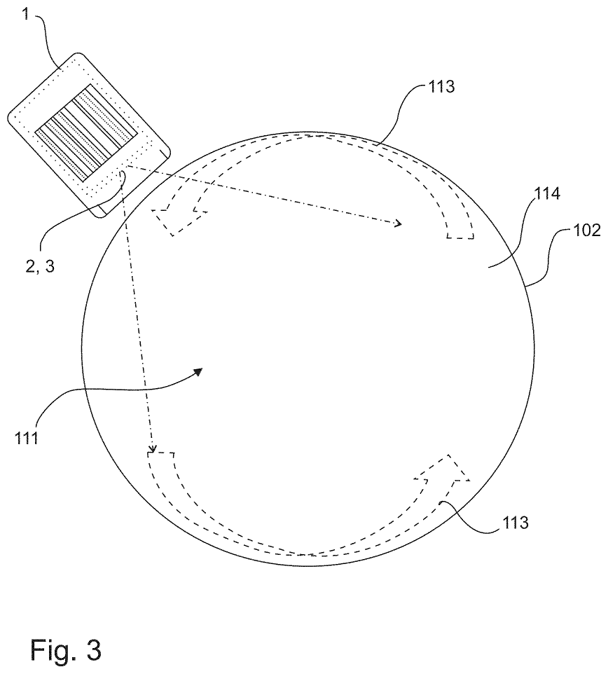

The container device 102 is also referred to as a suds container or a drum and is rotatably arranged in the housing 101 of the household appliance 100. The rotation is driven by a drive apparatus 112, typically an electric motor. A sealing device 110, which is also referred to as a door seal or drum seal, is provided between the housing 101 of the household appliance 100 and the container device 102. The closure apparatus 104 abuts this sealing device 110 in a closed state. The lighting apparatus 1 with integrated disinfection apparatus 2 is arranged in this sealing apparatus 110 in such a way that the first radiation source device 3 and the second radiation source device 4 can irradiate or illuminate at least a portion of the container device 102. The housing 7 is accordingly oriented such that the window or the front 5a of the carrier device 5 faces an interior of the container device 102. In FIG. 3, the container device 102 is shown in the form of a circular cylindrical suds container or a drum. Furthermore, a portion 111 of the container device 102 is shown, which is irradiated by the disinfection apparatus 2 or the first radiation source device 3. By rotating the container device 102 in accordance with the arrows 113 in FIG. 3, it can be ensured that the entire inner surface 114 of the container device 102 is irradiated or disinfected by the disinfection apparatus 2 or the first radiation source device 3. The porthole 104 is advantageously made of a material that is transparent to visible light, such as glass or plastics material. However, this material is advantageously not transparent to UV-C radiation. Thus, when the closure apparatus 104 is closed, no radiation from the first radiation source device 3 can penetrate out of the container device 102.

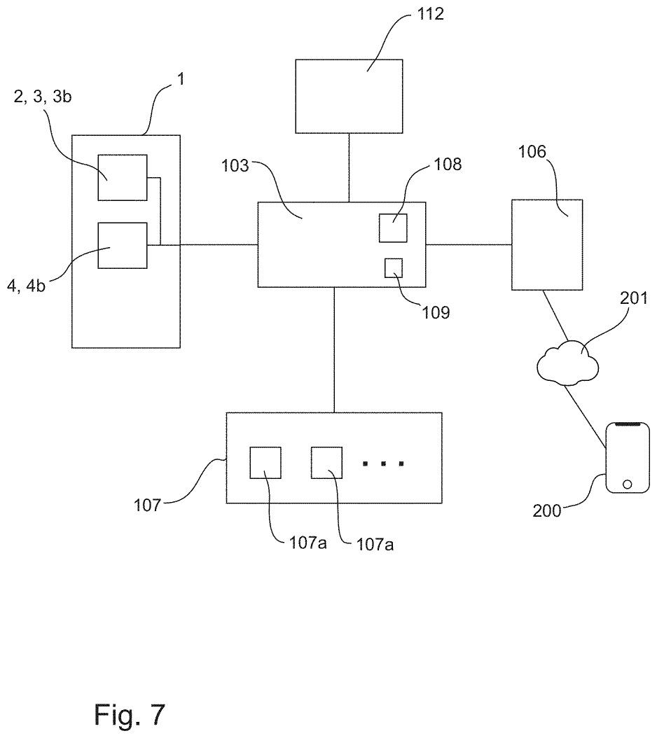

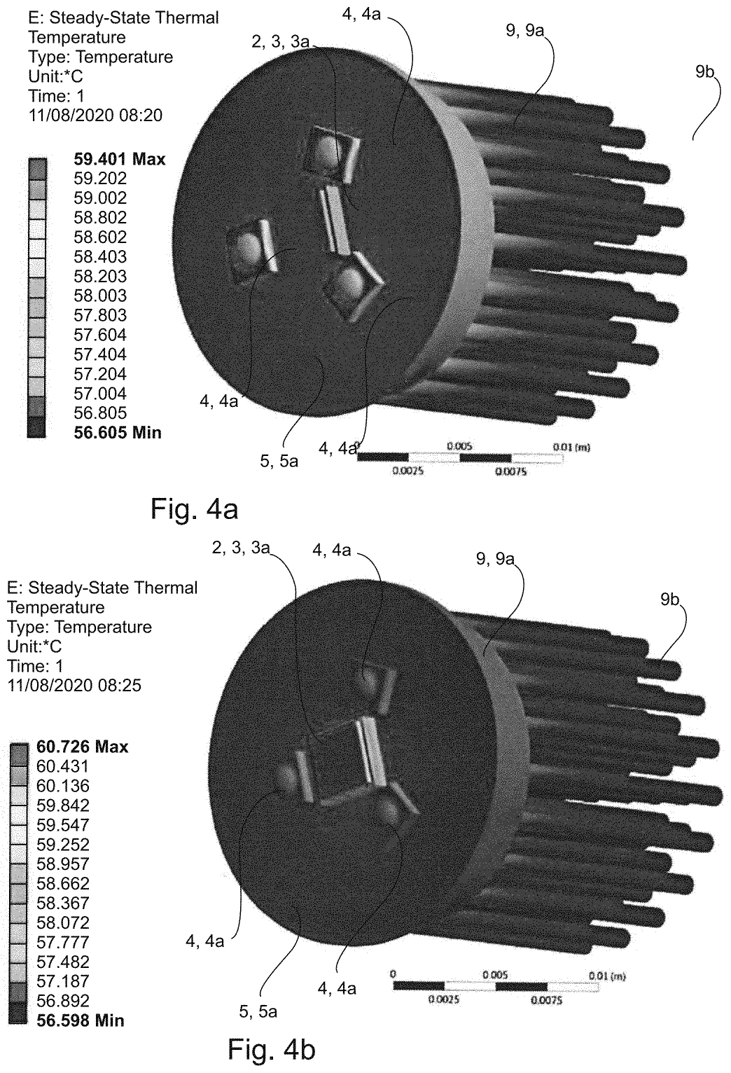

FIG. 7 is a schematic circuit diagram for a household appliance 100 having at least one lighting apparatus 1 with integrated disinfection apparatus 2. Accordingly, a control device 103 is provided which can be associated with the household appliance 100 or the lighting apparatus 1. A first driver device 3b n is preferably provided for the at least one radiation source 3a of the first radiation source device 3 in the form of a UV-C LED. A second driver device 4b is preferably provided for the at least one radiation source 4a of the second radiation source device 4 in the form of an LED. The driver devices 3b, 4b can be arranged inside the housing 7 or also outside the housing 7. The control device 103 is connected to the driver devices 3b, 4b for signalling purposes. The control device 103 is also connected to the drive device 112 of the container device 102 for signalling purposes.

In a first state of the lighting apparatus 1, the first radiation source device 3 or the disinfection apparatus 2 is activated. In a second state of the lighting apparatus 1, the first radiation source device 3 or the disinfection apparatus 2 is deactivated. In a third state of the lighting apparatus 1, the second radiation source device 4 is preferably activated. In a fourth state of the lighting apparatus 1, the second radiation source devices 4 are preferably deactivated. The defined first state and second state are independent of the defined third and fourth state. The lighting apparatus 1 can accordingly be in the first and third state or in the first and fourth state. Likewise, the lighting apparatus 1 can accordingly be in the second and third state or in the second and fourth state, in other words the first radiation source device 3 or the disinfection apparatus 2 can be activated or deactivated independently of the second radiation source device 4. The second radiation source device 4 can also be activated or deactivated independently of the first radiation source device 3 or the disinfection apparatus 2. The activation and/or deactivation of the first radiation source device 3 or the disinfection apparatus 2 is advantageously carried out by actuation of the first driver device 3b by the control device 103. The activation and/or deactivation of the second radiation source device 4 is advantageously carried out by actuation of the second driver device 4b by the control device 103. A first status signal relating to the first state of the lighting apparatus 1 can be received or generated by means of the control device 103. A second status signal relating to the second state of the lighting apparatus 1 can be received or generated by means of the control device 103. Furthermore, a third status signal relating to the third state of the lighting apparatus 1 can be received or generated by means of the control device 103. Further, a fourth status signal relating to the fourth state of the lighting apparatus 1 can be received or generated by means of the control device 103.

The control device 103 is connected for signalling purposes to at least one input device 106, by means of which the status signals can be generated. The input device 106 sends the status signals to the at least one control device 103, as a result of which the corresponding states are initiated. The input device 106 can preferably be operated manually. Accordingly, the input device 106 can comprise buttons and/or switches and/or a touch screen, for example. The input device 106 can preferably receive the first status signal and/or the second status signal from an external communications device 200 of a user. The connection between the external communications device and the input device is preferably a wireless connection 201. An external communications device 200 can be a smartphone, a tablet computer, a laptop or a similar device, for example. A corresponding wireless connection 201 can be, for example, an RFID (radio-frequency identification) connection, an NFC (near-field communication) connection, a Wi-Fi connection or a mobile phone connection. Of course, further wireless connections or wired connections can also be used.

The activation or deactivation of the lighting apparatus 1 can also advantageously take place automatically, however. For this purpose, the control device 103 itself preferably generates the status signal in each case. The status signals are subsequently processed by means of the control device 103 in such a way that the corresponding state is initiated. The at least one control device 103 preferably generates the status signals on the basis of sensor data from a sensor device 107. The sensor device 107 preferably comprises at least one sensor 107a which detects an opening and/or a closure of the closure apparatus 104. Since UV-C radiation can be dangerous for humans, it is advantageous if the control device 103 only activates the first radiation source device 3 or disinfects the container device 102 when a sensor signal is received by the sensor device 107 which indicates that the closure apparatus 102 of the household appliance 100 is closed. Activation of the second radiation source device 4 is also only carried out when a sensor signal is received by the control device 103 which indicates that the closure apparatus 102 of the household appliance 100 is open. The user is accordingly provided with lighting for the container device 102, which facilitates loading and unloading of the container device 102.

The sensor device 107 can comprise at least one sensor 107a which detects the loading state in the container device 102. Such a sensor 107a can be a weight sensor which detects the introduced weight of the items, for example. A sensor 107a in the form of a camera system, which can recognise a loading state, for example by image recognition, would also be conceivable. Advantageously, the first state could be triggered or the first radiation source device 3 could be activated when a load is detected, for example in the form of laundry to be cleaned in the case of a washing machine 100. Alternatively or cumulatively, the sensor device 107 comprises at least one sensor 107a which detects certain gases in the air in the container device 102. Such gases may be those which cause an unpleasant odour, for example. Preferably, the first state could be triggered or the radiation source device 3 could be activated when such a gas is detected. After the removal of the gas, the second state could then be triggered or the first radiation source device 3 could be deactivated.

The control device 103 preferably controls the drive apparatus 112 of the container device 102 in such a way that the container device 102 is rotated during the first state of the lighting apparatus or during the disinfection. As a result of the rotation of the container device 102, the entire inner wall of the container device 102 or the entire inner surface of the inner wall passes through the irradiated portion 111. After a rotation through a certain angle of rotation, the entire inner surface of the inner wall of the container device 102 would accordingly advantageously be exposed to the irradiation. In the first state, there is advantageously a predetermined number of rotations of the container device 102 in order to allow sufficient irradiation of the inner surface. The speed of rotation and the number of rotations are advantageously dependent on the radiation dose or the output power of the first radiation source device 3.

The first state can be activated when the container device 102 is empty. Accordingly, the container device 102, in particular the inner surfaces of the container device 02 and/or the air in the container device, is advantageously disinfected. The first state of the lighting apparatus 1 or the first radiation source device 3 or the disinfection apparatus 2 can be automatically activated at predetermined times and/or at regular intervals.

According to a further preferred embodiment, the first state is activated in a loaded state of the container device 102. Accordingly, objects to be washed in the container device 102, for example items of laundry, are advantageously disinfected. The first state can be activated before a washing program. Accordingly, the objects to be washed can already be disinfected before the washing cycle is carried out. Alternatively or cumulatively, the first state can also be activated after or during the execution of a washing cycle.

A status signal can preferably also be generated or received when a specific program of the household appliance is started, for example when a washing program is started. This can be done using the control device or also a further control device.

The status signals can be generated on the basis of a predetermined point in time or a predetermined time interval. The point in time of the activation of the states can thus advantageously be specified. The control device 103 comprises a memory device 109 in which particular sequence programs are stored. Sequence programs of this type can comprise the sequential actuation of specific devices, such as the first radiation source device 3, the second radiation source device 4, the drive apparatus 112, etc. Likewise, the intensity of the actuation of these devices can advantageously be provided in such a sequence program.

The applicant reserves the right to claim all the features disclosed in the application documents as essential to the invention, provided that these are novel, individually or in combination, over the prior art. It is further pointed out that features which in themselves can be advantageous have also been described in the individual drawings. A person skilled in the art will immediately recognise that a particular feature described in one drawing can also be advantageous without adopting further features from this drawing. A person skilled in the art will further recognise that advantages can also result from a combination of a plurality of features shown in individual or in different drawings.

### LIST OF REFERENCE SIGNS

1 Lighting apparatus
2 Disinfection apparatus
3 First radiation source device
3a Radiation source
3b First driver device
4 Second radiation source device
4a Radiation source
4b Second driver device
5 Carrier device
5a Front of the carrier device
5b Back of the carrier device
6 Cooling device
7 Housing
7 Window
8 Heat sink
9 Main body of the housing
10a Opening of the main body
10b Wall of the main body
11 Projection
12 Sealing device
13 Mounting projections
14 Compression element
100 Household appliance
101 Housing
102 Container device
103 Control device
104 Closure apparatus
105 Side wall
106 Input device
107 Sensor device
107a Sensor
108 Timer device
109 Memory device
110 Sealing apparatus
111 Irradiated portion of the container device
112 Drive apparatus
113 Arrow
114 Inner surface of the container device
200 Communication device
201 Wireless connection
X Longitudinal axis of the lighting apparatus
Y Width axis of the lighting apparatus
Z Height axis of the lighting apparatus
X′ Longitudinal axis of the household appliance
Y′ Width axis of the household appliance
Z′ Height axis of the household appliance

What is claimed is:

1. A lighting apparatus for a container device of a household appliance, in particular a washing machine, comprising:
at least one disinfection apparatus integrated in the lighting apparatus, wherein the at least one disinfection apparatus comprises a first radiation source device.

2. The lighting apparatus according to claim 1, wherein the first radiation source device comprises at least one radiation source, wherein the at least one radiation source is a light-emitting diode (LED), wherein the first radiation source device emits electromagnetic radiation having a wavelength in the range of 280 nm to 100 nm.

3. The lighting apparatus according to claim 1, wherein the lighting apparatus comprises a second radiation source device, wherein the second radiation source device emits electromagnetic radiation which is used to illuminate the container device, wherein the second radiation source device comprises at least one radiation source, wherein the at least one radiation source is a light-emitting diode (LED), wherein the second radiation source device emits electromagnetic radiation in a wavelength range between 400 nm and 700 nm.

4. The lighting apparatus according to claim 3, wherein the first radiation source device and the second radiation source device are arranged on a carrier device, wherein the carrier device is plate-like, wherein the carrier device has a substantially circular design.

5. The lighting apparatus according to claim 4, wherein the first radiation source device and the second radiation source device are arranged on the front of the carrier device, wherein at least one cooling device is arranged on the back of the carrier device, wherein the first radiation source device comprises only one radiation source which is arranged substantially centrally on the carrier device, wherein the second radiation source device comprises at least two radiation sources, wherein the at least two radiation sources are arranged radially at a distance from the radiation source of the first radiation source device on the carrier device.

6. The lighting apparatus according to claim 1, wherein the first radiation source device and the second radiation source device are arranged in a housing, wherein the housing comprises a window which is transparent both for the electromagnetic radiation of the first radiation source device and for the electromagnetic radiation of the second radiation source device, wherein the window consists of a quartz glass.

7. A household appliance comprising at least one lighting apparatus according to claim 1, wherein the household appliance comprises a housing and a container device provided therein.

8. The household appliance according to claim 7, wherein the housing of the household appliance comprises a closure apparatus by means of which the container device can be closed.

9. The household appliance according to claim 7, wherein at least one control device is provided, wherein in a first state of the lighting apparatus the first radiation source device is activated, wherein in a second state of the lighting apparatus the first radiation source device is deactivated, wherein in a third state of the lighting apparatus the second radiation source device is activated, wherein in a fourth state of the lighting apparatus the second radiation source device is deactivated, wherein the control device can receive or generate a first status signal relating to the first state of the lighting apparatus, wherein the control device can receive or generate a second status signal relating to the second state of the lighting apparatus, wherein the control device can receive or generate a third status signal relating to the third state of the lighting apparatus, wherein the control device can receive or generate a fourth status signal relating to the fourth state of the lighting apparatus.

10. The household appliance according to claim 9, wherein the at least one control device generates the first status signal and/or the second status signal on the basis of sensor data from a sensor device, wherein the sensor device comprises at least one sensor which detects an opening or a closure of the closure apparatus, wherein the sensor device comprises at least one sensor which detects the loading state in the container device, wherein the sensor device comprises at least one sensor which detects certain gases in the air of the container device.

11. The household appliance according to claim 7, wherein the household appliance is a washing machine, wherein the household appliance is a front-loading washing machine.

12. The household appliance according to claim 7, wherein the household appliance comprises a sealing apparatus which is arranged between the container device and the housing, wherein the lighting apparatus is arranged in the sealing apparatus.

13. The household appliance according to claim 7, wherein in the first state of the lighting apparatus, the first radiation source device irradiates a portion of the container device, wherein the household appliance comprises a drive apparatus, by means of which a rotation of the container device can be driven, wherein in the first state a rotation of the container device takes place.

14. The household appliance according to claim 10, wherein the first state of the lighting apparatus can only be initiated after at least one sensor of the sensor device has detected the closure of the closure apparatus.

15. The household appliance according to claim 7, wherein the first state is activated in an empty state of the container device, wherein the first state is activated before a washing program, wherein the first state is activated in a loaded state of the container device, wherein the first state is activated before a washing program.
